(19) United States
(12) Plant Patent Application Publication
(10) Pub. No.: US 2006/0085876 P1
(43) Pub. Date: Apr. 20, 2006

(54) Pistachio plant called 'PETE I'

(76) Inventor: David R. Petersen, Chico, CA (US)

Correspondence Address: CHRISTIE, PARKER & HALE, LLP, PO BOX 7068, PASADENA, CA, 91109-7068, US

(21) Appl. No.: 10/966,796

(22) Filed: Oct. 14, 2004

(51) Int. Cl. A01H 5/00

(52) U.S. Cl. PLT/152

(57) ABSTRACT

A new and distinct pistachio tree named ‘PETE I’, characterized by a very white shell nut with a 90% or higher split ratio and a large nut size (18 to 20 nuts per ounce), nut meat color brilliant purple on the exterior with a dark green interior, a very high resistance to botryosphaeria fungus, large, dark green leaves and an early bloom.

### BOTANICAL CLASSIFICATION

Pistachio sp.

### CULTIVAR NAME

‘PETE I’

### BACKGROUND OF THE INVENTION

The present invention relates to a new and distinct variety of pistachio tree, propagated from a T-budding sport.

Naturally occurring sports were collected, budded onto existing Pioneer II rootstock, and analyzed for resistance to the fungus botryosphaeria. One sport was selected for its high resistance to the fungus and designated ‘PETE I.’ The parents of ‘PETE I’ are unknown.

### SUMMARY OF THE INVENTION

The ‘PETE I’ variety is characterized by a very white shell nut with a 90% or higher split ratio and a large nut size (18 to 20 nuts per ounce). Nut meat color is brilliant purple on the exterior with a dark green interior.

‘PETE I’ is further characterized by a very high resistance to botryosphaeria, a major pistachio fungus, when compared to ‘Kerman’, ‘Kalleh Ghoochi’, and other pistachio varieties.

‘PETE I’ has a very upright growth habit. In the pre-harvest years (1 to 6 years), vegetative growth exceeds four feet per year with flower buds appearing in the fourth year. Leaves are deep green in color, compound with three to five, average of three, leaflets. During years 1 to 3, leaflets are noticeably larger, sometimes exceeding six inches in length and width. ‘PETE I’ trees have very dense foliage during the early years, becoming more aerated with maturity.

‘PETE I’ pollinates early compared to ‘Kerman’, typically 5 to 7 days earlier. First flowers are receptive in late March compared to early April for ‘Kerman’. Flowers are consistent in size and conformation, with an inverted Christmas tree appearance. Early pollination gives ‘PETE I’ a competitive advantage over varieties like ‘Kerman’ in that nuts of ‘PETE I’ will be ready for market earlier than those of ‘Kerman’ and other varieties.

Asexual reproduction by budding onto a Pioneer Gold II rootstock at Butte County, Calif. shows that the foregoing and other distinguishing characteristics come true to form and are established and transmitted through succeeding propagation. Other known forms of asexual reproduction for pistachios may also be employed.

### BRIEF DESCRIPTION OF ILLUSTRATIONS

The accompanying photographic illustrations show typical specimens of vegetative growth of the new variety, in color as nearly true as it is reasonably possible to make in a color illustration of this character.

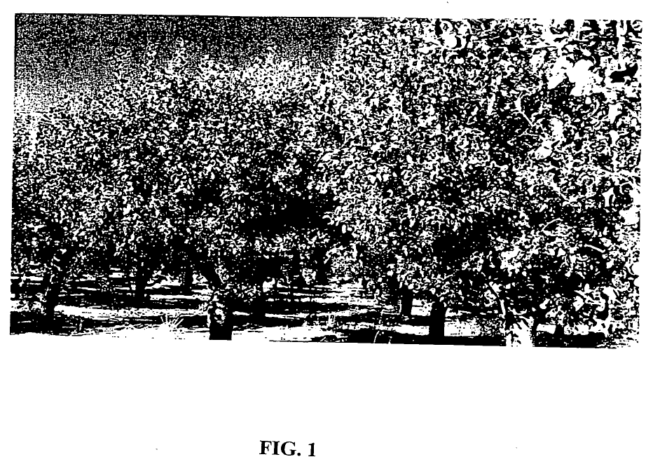

FIG. 1 shows an 18 year old pistachio tree of the new variety.

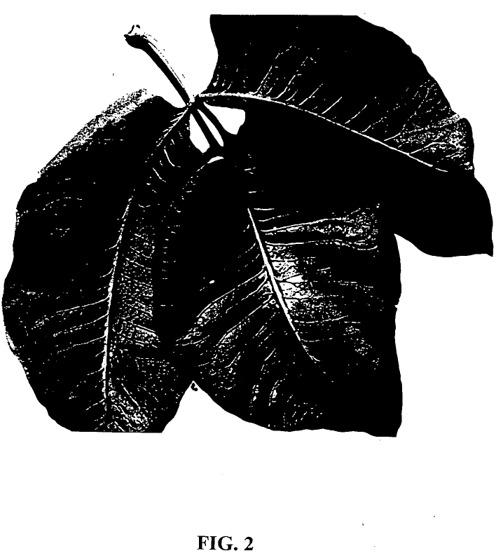

FIG. 2 shows a close-up of a leaf of the new variety.

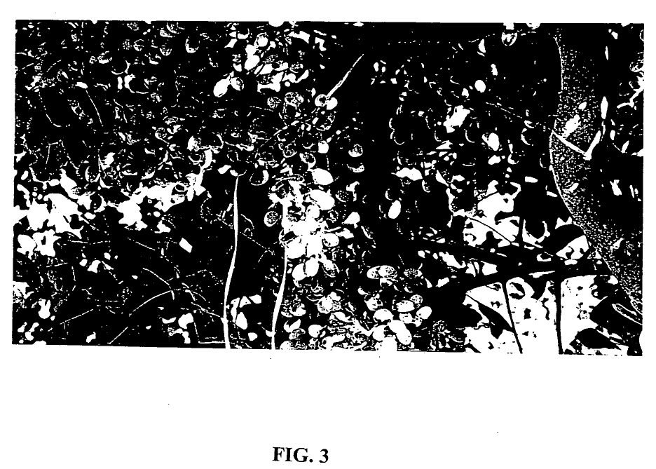

FIG. 3 shows a close-up of clusters of nuts of the new variety.

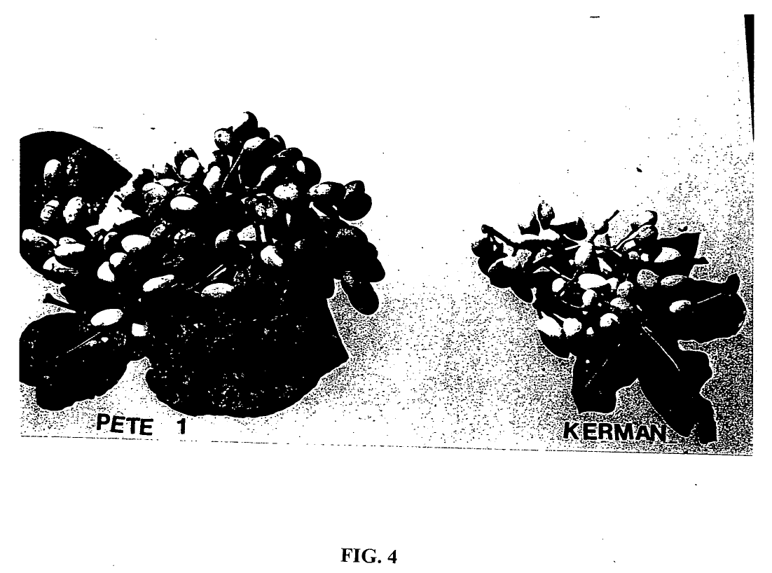

FIG. 4 shows a close-up comparison of the leaves and nut clusters of the new variety and ‘Kerman’.

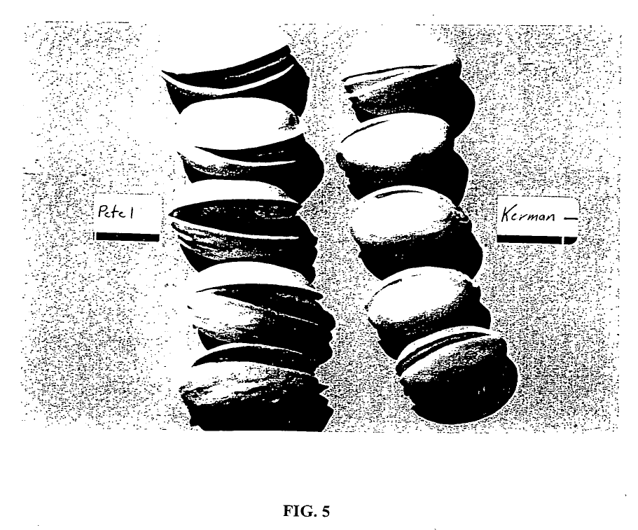

FIG. 5 shows a close-up comparison of nuts of the new variety and ‘Kerman’.

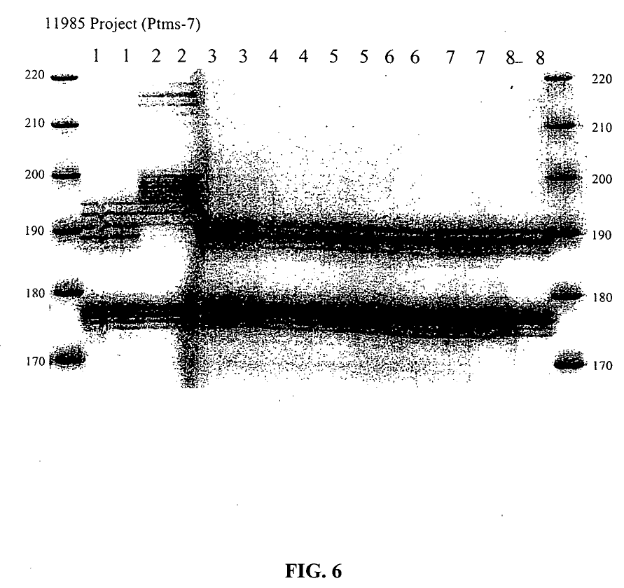

FIG. 6 shows an autoradiogram showing the results of PCR analysis of leaf DNA from several varieties tested with the SSR marker Ptms-7. Lane 1: ‘Calagucci’ (an Iranian pistachio variety); lane 2: ‘Peters’ (standard United States male pistachio); lanes 3 and 4: ‘PETE I’; lane 5: ‘Kerman’ (standard United States female pistachio); lane 6: ‘Kerman’ (from USDA Depository at U. C. Davis); lane 7: ‘Kerman’ (from southern California); lane 8: ‘Kerman’ (from southwestern California).

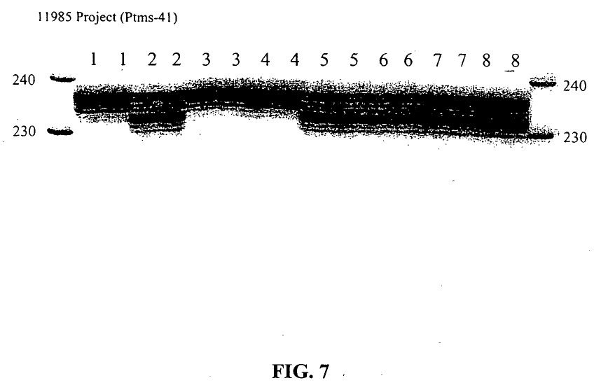

FIG. 7 shows an autoradiogram showing the results of PCR analysis of leaf DNA from several varieties tested with the SSR marker Ptms-41. Lane 1: ‘Calagucci’ (an Iranian pistachio variety); lane 2: ‘Peters’ (standard United States male pistachio); lanes 3 and 4: ‘PETE I’; lane 5: ‘Kerman’ (standard United States female pistachio); lane 6: ‘Kerman’ (from USDA Depository at U. C. Davis); lane 7: ‘Kerman’ (from southern California); lane 8: ‘Kerman’ (from southwestern California).

### COMPARISON TO ‘KERMAN’

The closest commercial equivalent to ‘PETE I’ of which I am aware is ‘Kerman’. The new variety differs from ‘Kerman’ in the following characteristics:

| | ‘PETE I’ | ‘Kerman’ |
|---|---|---|
| Nuts | White shell with brilliant | White shell with a pale green |
| | green meat. Clusters are | meat. Clusters are less dense |
| | spectacular in density | (10 to 15 nuts/cluster). Open - |
| | (20 to 30 nuts/cluster). | splits 75-80% with a higher |
| | Open - splits 90% or more | degree of non-splits. |
| | with a high percentage of | |
| | 20 to 30 nuts/cluster. | |
| Disease | High degree of resistance | Very low resistance to |
| Resis- | to botryosphaeria. In | botryosphaeria, especially in |
| tance | mature trees, average | areas of heavy rainfall. Average |
| | occurrence of | occurrence of botryosphaeria in |
| | botryosphaeria in | ‘Kerman’ is about 100 |
| | ‘PETE I’ is about 5 | strikes/tree, compared to 5 |
| | strikes/tree, compared to | strikes/tree for ‘PETE I’. |
| | 100 strikes/tree for | |
| | ‘Kerman’. | |
| Growth | Upright growth with large, | Upright growth with average size |
| Habits | dark green leaves; | leaves; usually 5 leaflets. |
| | typically 3 leaflets. | Grows 3-4 feet/year with flower |
| | Grows 3-4 feet/year | buds appearing in the fifth year. |
| | with flower buds appearing | |
| | in the 4th year. | |
| Polli- | Blooms in late March | Blooms in early April (typically |
| nation | through early April | March 30 through April 15, in |
| | (typically, March 25 | Butte county, California). |
| | through April 10, in | |
| | Butte county, California). | |

### DESCRIPTION OF THE NEW VARIETY

Referring now specifically to the new and distinct variety of pistachio tree, the following is a detailed description of 18 year old plants with color description where indicated by reference to The Royal Horticultural Society Colour Chart, except where common terms of color definition are employed.

The new variety of pistachio tree as herein described may vary in slight detail due to climatic, soil and cultural conditions under which the variety may be grown, the present description being of the variety as grown at Butte County, Calif.
Plant:
   Shape.—Symmetric canopy as shown in FIG. 1, about 16 feet in diameter.
   Height.—About 17 to 18 feet at maturity.
   Growing habit.—Before maturity, growth is cane in appearance, growing approximately 3 to 4 feet per year. At maturity, growth is 1 to 2 feet per year.
   Branching habit.—Established trunk with primary scaffolds starting in about the 40-inch range. Growth is cane-like in vegetation with smaller fruiting spears.
   Lateral branches.—Approximately 6 to 8 in number; about 4 feet in length, 6 to 7 inches in diameter at maturity.
   Disease resistance.—High resistance to botryosphaeria fungus.
Foliage:
   Leaf arrangement.—Compound, three to five leaflets, with an average of three, as shown in FIG. 2.
   Color.—Green, near 138A.
   Leaflet size/shape.—Length: about 4 to 6 inches; width: about 4 to 6 inches; shape oval.
   Leaflet base.—Broadly cuneate.
   Leaflet apex.—Obtuse.
   Petiole length.—About 4 to 6 inches.
   Petiole color.—Light yellow-green, near 147C-D.
Bloom:
   Form.—Inverted Christmas tree appearance.
   Borne.—Typically 3 to 7 flowers per lateral stem.
   Flowering season.—Late March through mid-April.
   Flower diameter.—Base: about 2 to 3 inches; tip: about ½-inch.
   Flower depth.—About 6 inches at the end of bloom.
Reproductive organs:
   Pistil.—Yellow.
Nuts: See FIGS. 3-5.
   Shell color.—Very white, near 155C-D.
   Nut meat color.—Exterior: brilliant purple, near 61B; interior: dark green.
   Split ratio.—90% or higher
   Number per ounce.—About 18 to 20.

PCR analysis of DNA from leaves of ‘PETE 1’ show unique restriction fragments, when compared to other pistachio varieties, for markers Ptms-7 and Ptms-41. See FIGS. 6-7.

We claim:

1. A new and distinct variety of pistachio tree, identified as ‘PETE 1’, substantially as shown and described.
